(19) United States
(12) Patent Application Publication
(10) Pub. No.: US 2021/0110287 A1
(43) Pub. Date: Apr. 15, 2021

(54) Causal Reasoning and Counterfactual Probabilistic Programming Framework Using Approximate Inference

(71) Applicant: Babylon Partners Limited, London (GB)

(72) Inventors: Iurii Perov, London (GB); Logan Christopher Sherwin Graham, London (GB); Konstantinos Gourgoulias, London (GB); Jonathan George Richens, London (GB); Ciarán Mark Lee, London (GB); Adam Philip Baker, London (GB); Saurabh Johri, London (GB)

(21) Appl. No.: 16/944,512

(22) Filed: Jul. 31, 2020

(30) Foreign Application Priority Data
Oct. 15, 2019 (GB) 1914903.8

(51) Int. Cl. G06N 5/04; G06N 7/00

(52) U.S. Cl. CPC G06N 5/04; G06N 7/005

(57) ABSTRACT

A computer implemented method of performing inference on a generative model,
wherein the generative model in a probabilistic program form, said probabilistic program form defining variables and probabilistic relationships between variables, the method comprising:
providing at least one of observations or interventions to the generative model;
selecting an inference method, wherein the inference method is selected from one of: observational inference, interventional inference or counterfactual inference;
performing the selected inference method using an approximate inference method on the generative model; and
outputting a predicted outcome from the results of the inference;
wherein approximate inference is performed by inputting an inference query and the model, observations, interventions and inference query are provided as independent parameters such that they can be iterated over and varied independently of each other.

### FIELD

Embodiments of the present invention relate to the field of computer implemented determination methods and systems.

### BACKGROUND

Probabilistic programming languages (PPL) are used to define probabilistic programming frameworks (PPF). PPLs are used to formalise knowledge about the world and for reasoning and decision-making. They have been successfully applied to problems in a wide range of real-life applications including information technology, engineering, systems biology and medicine, among others.

Probabilistic Graphical Models (PGMS) can be expressed in a PPL and they provide a natural framework for expressing the probabilistic relationships between random variables in numerous fields across the natural sciences. Bayesian networks, a directed form of graphical model, have been used extensively in medicine, to capture causal relationships between entities such as risk-factors, diseases and symptoms, and to facilitate medical decision-making tasks such as treatment and recovery analysis and disease diagnosis. Key to decision-making is the process of performing probabilistic inference to update one's prior beliefs about the likelihood of a set of variables of interest, based on the observation of new evidence,

PPFs are designed to allow out-of-the-box, efficient inference in PGMs written in PPLs.

### BRIEF DESCRIPTION OF THE FIGURES

FIG. 1 is a schematic of a system in accordance with an embodiment;

FIG. 2 is a diagram of a probabilistic graphical model of the type that can be used with the system of FIG. 1;

FIG. 3 is a flow chart for explaining the counterfactual diagnosis method in accordance with an embodiment;

FIG. 4a is a diagram of a simple PGM; FIG. 4b is a diagram of a simple PGM represented as a structured causal model and FIG. 4c is a variation on the structured causal model of FIG. 4b

FIG. 5 is a more detailed flow chart for counterfactual inference on a PGM;

FIG. 6 is a flow chart for counterfactual inference on the PGM of FIG. 4b with optimisations including static analysis;

FIG. 7 is a schematic of a system in accordance with an embodiment;

FIG. 8 is a schematic of a system in accordance with another embodiment.

### DETAILED DESCRIPTION

In an embodiment, a computer implemented method of performing inference on a generative model is provided,
wherein defining the generative model in a probabilistic program form, said probabilistic program form defining variables and probabilistic relationships between variables, the method comprising:
providing at least one of the observations or the interventions to the generative model;
selecting an inference method, wherein the inference method is selected from one of; observational inference, interventional inference or counterfactual inference;
performing the selected inference method using an approximate inference method on the generative model; and
outputting a predicted outcome from the results of the inference;
wherein approximate inference is performed by inputting an inference query and the model, observations, interventions and inference query, are provided as independent parameters to the inference engine such that they can be iterated over and varied independently of each other.

Generative models, such as probabilistic graphical models now form the backbone of many decision and medical systems. However, they require significant computing resources such as processor capacity that makes real time exact inference impossible. The disclosed systems and methods solve this technical problem with a technical solution, namely by providing efficient abstraction and separation of the steps of (1) defining a generative model, (2) applying observations to the model and (3) running statistical inference on the model to provide an estimate of the posterior probabilities. The above therefore enable the system to produce answers using such new approximate inference with the accuracy comparable to using exact or already existing approximate inference techniques, but in a fraction of the time and with a reduction in the processing required.

A PPL consists of a set of elementary random predicates/procedures (ERP) and a deterministic program written in a host language.

The above method allows the same generative model to be used regardless of whether counterfactual, observational or interventional interference is selected. Thus, just one model needs to be stored and the nodes of this model are instantiated as required.

For observational inference, the likelihoods of observed nodes is incorporated into the weights. For interventional inference, an observational inference is performed on the modified models (with interventions). For counterfactual inference, firstly the posterior version of the model is produced by doing observational inference, and then the intervention is performed on that model.

In the above embodiment, the model in its entirety is considered to be a single parameter. Thus, when approximate inference is performed, all four parameters, i.e. the model, observations, interventions and inference query are provided as independent parameters to the inference engine such that they can be iterated over and varied independently of each other.

In an embodiment, the above allows approximate inference to be performed in an inference engine and each inference query is fully performed as just one single request to the inference engine. Thus, the inference engine would receive a query (i.e. to calculate the likelihood of a treatment working, likelihood of a disease given observations etc). This is to be contrasted with methods where multiple calls will need to be sent to the inference engine.

The generative model may be provided on a first server and the inference query may be input at a location separate from the first server, wherein the inference query is sent as a single request from the said location to the first server and the server returns the result of the inference as a single message to the said location. The inference query may input via a mobile device, for example, a phone, tablet, laptop etc. The simplicity of the single query reduces the amount of information that needs to be sent.

In an embodiment, the result of the inference can be expressed as expected values of the variables of interest.

In an embodiment, the generative model is a structured causal model where noise is modelled as a separate variable. In a structured causal model, the generative model expresses noise as an explicit random variable. In another embodiment, a generative model is a graphical model or any possible directed generative model.

In an embodiment, the approximate inference method is importance sampling.

In an embodiment, the inference engine supports observable probabilistic procedures that propagate the observation value to explicit noise variables that represent part of the execution of the trace for efficient counterfactual inference, here, the noise variables are represented explicitly and noise variables constitute the part of the joint posterior of the abduction step.

In an embodiment performing observational inference comprises: weighting the prior space of the generative model by the likelihood that is calculated on the observed probabilistic procedures. Observed probabilistic procedures are not sampled; instead, their likelihood is computed and incorporated into the weights given the observations.

In an embodiment, performing interventional inference comprises: having a representation of the generative model with intervened variables, and then performing the observational inference as above.

In an embodiment, performing counterfactual inference comprises: performing the observational inference as above, then, given that model, performing intervention on a representation of that model (e.g. in the form of samples from importance sampling algorithm) and predicting the variables of interest. During the observational step, it is important to represent the noise distributions of observed probabilistic procedures since they must influence the predicted variables after the intervention.

In a further embodiment, the method further comprises performing statics analysis on the generative model, and observations and interventions, given inference query types, to optimise the inference method. In yet further embodiments, dynamic analysis may also be performed.

In a further embodiment a system is provided for performing inference on a generative model,
the system comprising a processor and a memory, the generative model being stored in a probabilistic program form in said memory, said probabilistic program form defining variables and probabilistic relationships between variables, the processor being configured to:
provide at least one of observations or interventions to the generative model;
allow selection of an inference method, wherein the inference method is selected from one of: observational inference, interventional inference or counterfactual inference;
perform the selected inference method using an approximate inference method on the generative model; and
output a predicted outcome from the results of the inference;
wherein approximate inference is performed by inputting an inference query and the model, observations, interventions and inference query are provided as independent parameters such that they can be iterated over and varied independently of each other.

The user may select the type of inference themselves or the processor may be adapted to determine the type of inference dependent on the question to be answered. For example, if the user provided a question such as “I have a headache and I want to know what is wrong” then observation inference would be selected. If the user provided a question such as “Would my risk of a heart attack be lower if I took statins” then interventional inference might be selected. However, if for example, the user was a smoker and provided the question “what if I stopped smoking” then counterfactual inference might be more appropriate. Whereas it is possible for the query to be input via natural language, in other embodiments, the system supports queries that are not via natural language (e.g. queries where data and data structures are passed to the model).

To give context to one possible use of a system in accordance with an embodiment, an example will be discussed in relation to the medical field. However, embodiments described herein can be applied to any causal reasoning queries on a generative model.

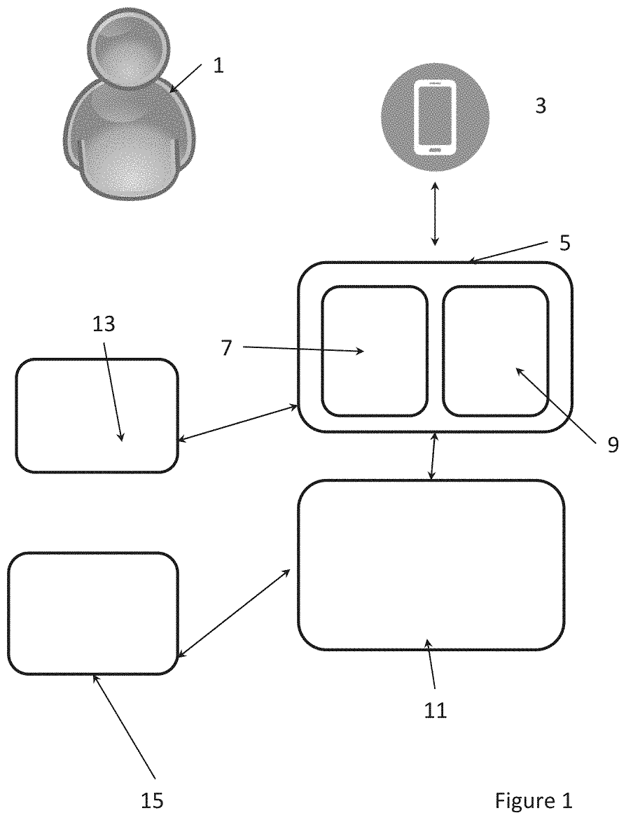

FIG. 1 is a schematic of a system. In one embodiment, a user 1 communicates with the system via a mobile phone 3. However, any device could be used, which is capable of communicating information over a computer network, for example, a laptop, tablet computer, information point, fixed computer etc.

The mobile phone 3 will communicate with interface 5. Interface 5 has 2 primary functions, the first function 7 is to take the words uttered by the user and turn them into a form that can be understood by the inference engine 11. The second function 9 is to take the output of the inference engine 11 and to send this back to the user's mobile phone 3.

In some embodiments, Natural Language Processing (NLP) is used in the interface 5. NLP helps computers interpret, understand, and then use everyday human language and language patterns. It breaks both speech and text down into shorter components and interprets these more manageable blocks to understand what each individual component means and how it contributes to the overall meaning, linking the occurrence of medical terms to the Knowledge Graph. Through NLP it is possible to transcribe consultations, summarise clinical records and chat with users in a more natural, human way.

However, simply understanding how users express their evidence (e.g. their risk factors, treatment, or symptoms) is not enough to identify and provide reasons about the underlying set of variables of interest (e.g. treatments or diseases). For this, the inference engine 11 is used. The inference engine is a powerful set of machine learning systems, capable of reasoning on a space of >100s of billions of combinations of nodes, to calculate the posterior, interventional and counterfactual queries. The inference engine can provide reasoning efficiently, at scale, to bring healthcare to millions.

In an embodiment, the Knowledge Graph 13 is a large structured medical knowledge base. It captures human knowledge on modern medicine encoded for machines. This is used to allows the above components to speak to each other. The Knowledge Graph keeps track of the meaning behind medical terminology across different medical systems and different languages.

In an embodiment, the patient data is stored using a so-called user graph 15.

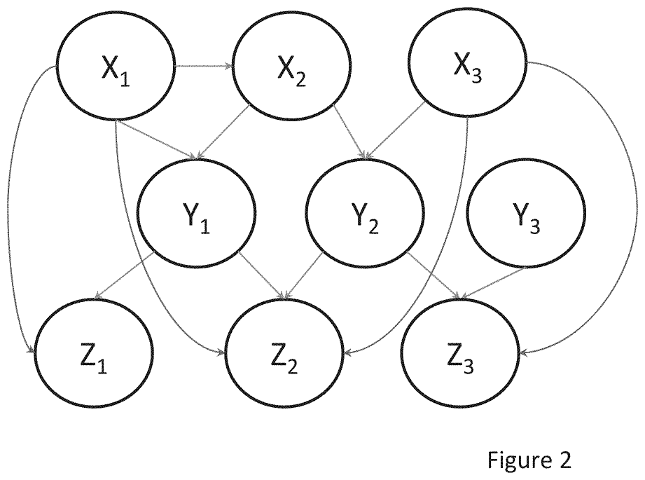

In an embodiment, the inference engine 11 comprises a generative model which may be a probabilistic graphical model or any type of probabilistic framework. FIG. 2 is a depiction of a probabilistic graphical model of the type that may be used in the inference engine 11 of FIG. 1.

Clinical diagnostic models (CDMs) can be probabilistic graphical models (PGMs) that model hundreds of categorical and continuous nodes (e.g, risk factors, treatments, recovery, symptoms, diseases, etc.).

The graphical model provides a natural framework for expressing probabilistic relationships between random variables, to facilitate causal modelling and decision making. In the model of FIG. 2, when applied to medical domain, there are different dependencies between different nodes on different levels. There are prior probabilities and conditional probabilities that describe the “strength” (probability) of connections.

One of examples of probabilistic generative models with structure as in FIG. 2, which are used in healthcare and on which someone can perform counterfactual queries for medical analysis, is the model of treatment and recovery. For such a model, the “effect of treatment on the treated” counterfactual query is computed. Such a model can be implemented as a probabilistic program and a counterfactual query can be computed on it. Such model is used to analyse the quality of treatments and their effects on recovery for populations that require treatments. Nodes in FIG. 2 for such model will have meaning as follows: nodes X_i will be the environmental variables (e.g. gender of people or socioeconomic factors) that can influence both the probability of treatments and probabilities of the recovery, nodes Y_i will be treatments (e.g. drugs taken or medical procedures), and nodes Z_i will be indicator variables for recovery of problems (e.g. pain or disease recovery). A standard counterfactual query to be applied to such model is the “average effect of treatment on the treated” (called ETT): it is a difference (subtraction) between the average recovery Z_i of a person who is treated P (Recovery_i=True|Treatment_j=True) and the average probability of how likely the recovery Z_i of a person who was originally treated is, if they had not been treated (i.e. intervened, where the intervention is expressed by the do operator) P(Recovery_i=True|Treatment_j=True, do(Treatment_j=False)). While the first term of the ETT is a standard observational query, the second part of the ETT is a counterfactual query because it asks a counter factual question for a group of people who are treated: what would happen if they, who are treated, had not been treated? Estimating the ETT is important in healthcare (as well as in other fields) because it allows one to understand whether the treatment is actually effective by itself or not. If the ETT is high, then the treatment is effective. If the ETT is around 0, then the treatment is not very effective. If the ETT is negative, then the treatment might be actually harmful. Note that the example above is only one possible example and it does not restrict the set of models and queries that the user can express and evaluate with the embodiments. Other queries are possible, including observational (posterior) and interventional queries; as well as counterfactual queries where you intervene on any variable or set of variables regardless of whether it is observed.

The embodiments described herein relate to defining or creating the model, applying the observations to the model and the inference engine.

In an embodiment, in use, a user 1 may input their evidence via interface 5. The interface may be adapted to ask the user 1 specific questions. Alternately, the user may just simply enter free text. The user's evidence may be derived from the user's records held in a user graph 15. Therefore, once the user identified themselves, data about the patient could be accessed via the system.

In further embodiments, follow-up questions may be asked by the interface 5. Flow this is achieved will be explained later. First, it will be assumed that the user provides all possible information (evidence and intervention, along with the query type) to the system at the start of the process.

The evidence will be taken to be the presence or absence of all known evidence nodes. For nodes where the user has been unable to provide a response, these will assume to be unknown.

Next, this evidence is passed to the inference engine 11. In an embodiment, inference engine 11 is capable of performing any type of inference (i.e. observational, interventional or counterfactual inference), on PGM of FIG. 2.

Due to the size of the PGM, it is not possible to perform exact inference in a realistic timescale. Therefore, the inference engine 11 performs approximate inference. When performing approximate inference, the inference engine 11 requires an approximation of the probability distributions within the PGM to act as proposals for the sampling.

A PGM can be defined using a probabilistic programming language (PPL) in a probabilistic programming framework. In a probabilistic program nodes and edges are used to define a distribution p(x, y). Here, x are the latent variables and y are the observations.

Inference can be performed by performing the following queries:
1. Observational queries
2. Interventional queries
3. Counterfactual queries

A background on these inference methods is given below.

Observational Inference

Observational inference is also referred to as posterior inference. Given a model M with latent variables X and observed variables Y such that we can define P(X, Y), an observational query may be defined as P(X|Y) (or, generally, P(T ⊆ X, K ⊆ Y), where T are variables of interest and K are partially observed nodes).

Interventional Inference

Interventional query, P(W|do(D=d),E) where W, E, D ⊆ X ∪ Y and dare the values that D takes. W are the values of interest for the query, and E are the observations.

To perform an interventional query, firstly all incoming edges to variables D are removed. The variables D are set to specific values a and by doing so a new model is generated. Then, the posterior inference is performed in this new model M′ such that the posterior P_{M′}(W|E) can be calculated.

In other words, first the intervention is done on the model modifying it into a new model, and then the posterior inference is done in this new model. That is, we firstly modify the model to account for the intervention, and then do the posterior inference in that model.

Counterfactual Inference

A counterfactual query is of the form P(W′|E,do(D=d)) where W are any variables and W′ are their counterfactual parts in the counterfactual query.

Counterfactual queries are different from observational and interventional queries. They consist of three steps (for reference, e.g. see Causality by Pearl, 2nd edition, page 206):
1. Abduction; update P(W) by the evidence E to obtain the posterior P(W|E) i.e. model M′.
2. Action: modify the model M′ by the action do(D=d).
3. Prediction: use the modified model M″ to compute the consequence of the counterfactual P_{M″}(W′) in the probability space defined by the posterior.

In other words, firstly the posterior inference is performed (hence calculating/deriving the posterior probability space under the evidence), and then the intervention/action is performed in that space.

The first step, updating the distribution over latents and storing the resulting probabilities, requires a large amount of computational resources and memory—especially as conditioning on evidence induces correlations between initially uncorrelated latents. Moreover, as this step has to be repeated for every new counterfactual query, such computational resources are continually required. Therefore, exact abduction is intractable for large models with significant tree-width such as medical networks.

As noted above, a PGM can be defined using a probabilistic programming language (PPL) in a probabilistic programming framework.

The purpose of a probabilistic program is to implicitly specify a probabilistic generative model. In an embodiment, probabilistic program systems will be considered to be systems such that:

(1) the ability to define a probabilistic generative model in a form of a program,

(2) the ability to condition values of unknown variables in a program such that this allows data from real world observations to be incorporated into a probabilistic program and infer the posterior distribution over those variables. In some probabilistic programs, this is achieved via observe statements.

Probabilistic programs are capable of calling on a library of probabilistic distributions that allow variables to be generated from the distributions in a model definition step. Such distributions can be selected from, but not limited to Bernoulli; Gaussian; Categorical etc:

Examples of possible sampling steps are:

Variable1=Bernoulli(μ)

Variable2=Gaussian(μ,σ)

Etc

In the above, Variable2 sampled from the Normal distribution and μ and σ are the mean and standard deviation respectively.

Probabilistic programs can be used to represent probabilistic graphical models (PGM) which use graphs to denote conditional dependencies between random variables. The probability distributions of a PGM can be encoded in a probabilistic program by, for example, by encoding each distribution from which values are to be drawn. Different values for the parameters of a distribution can be set dependent on the variable of an earlier distribution in the probabilistic program. Thus, it is possible to encode complex PGMs.

As noted above, a probabilistic program can also be used to condition values of the variables. This can be used to incorporate real world observations. For example, in some syntax the command “Observe” will allow the output to only consider variables that agree with some real world observation.

For example

Observe (c=1)

Would block all runs (samples) where the variable c≠1.

The inference stage allows an implicit representation of a posterior multi variable probability distribution to be defined. The inference stage may use an exact inference approach, for example, junction tree algorithm etc. Approximate inference is also possible as discussed herein using, for example, importance sampling.

When performing approximate inference for a probabilistic program, the inference stage would often require many samples to be produced by the sampling stage. Each sample can be viewed as a thread or run through the PGM where during the collection of each sample, variables are stored in memory or accumulated in aggregated statistics (e.g. mean or variance). By using a data driven proposal, the number of samples required can be reduced and therefore the number of accesses within the memory and the number of calls to a processor to perform the sampling process are reduced. Further, the closer the proposal distribution to the target distribution, the smaller generally the number of samples required.

Importance Sampling

Importance sampling estimates properties of a particular distribution, while only having samples generated from a different distribution than the distribution of interest.

In more detail, importance sampling is a Monte Carlo method useful in estimating integrals of the form ∫f(x)p(x). dx but where it is not possible to sample from p directly. The idea is to repeatedly sample from a proposal distribution q and, for each sample x, compute a weight given by w(x)=p(x)/q(x). It is possible to use the samples and weights to estimate the expectation of interest by Σᵢ₌₁ⁿ w(xᵢ)f(xᵢ). The proposal distribution is chosen by the user. If only the unnormalised target is available then it is called self-normalised importance sampling. This method produces a biased but consistent estimator of the expectation of interest.

In importance sampling as applied to inference, the posterior can be calculated by the virtue of sampling samples from a prior/proposal distribution and accumulating the prior+proposal+likelihood probabilities into the weights, and then calculating the statistics of interest using those samples plus their weights.

Samples (each sample contains values for all X and Y):

sᵢ˜proposal Q(X)

Weights:

$${w}_{i} = \frac{prior ( X )}{proposal ( X )} \times likelihood ( Y \vert X )$$

In most cases, X is a vector and it can be sampled forward from the model element by element.

After that, different statistics can be computed, for an example an expectation with some function f:

$${E}_{X \vert Y} [ f ( X ) ] = \frac{Σ f ( {s}_{i} ) \times {w}_{i}}{Σ {w}_{i}}$$

The presented methods involve self-normalising importance sampling. Generally, performing self-normalisation to estimate the normalised posterior is necessary to represent the real posterior. Otherwise non-normalised quantities are received which, while they can be used to compare them relatively between each other, don't represent the real posterior.

Importance sampling is beneficial in inference where it is difficult or computationally expensive to generate samples from the real posterior. Sampling from the proposal can make the inference more efficient and improve the resource-burden of computing inference queries.

Importance sampling can also make the samples more relevant to the evidence (depending on the proposal used). As a result, it can make the abduction step more efficient as fewer samples are needed to be generated. Furthermore, it can reduce the variance in the estimate/prediction step as the samples are more relevant.

Using probabilistic programming also makes executing the inference more efficient as it provides automatised inference methods. In probabilistic programming with importance sampling, each variable xᵢ ∈ X, yᵢ ∈ Y is represented as an elementary procedure. To sample a sample sᵢ of the whole probabilistic program, the program is evaluated forward and in the process:
1. For latent (unobserved) variables, each variable value is sampled from its proposal, and the likelihood of prior and proposal are incorporated into the weight.
2. For observed variables, we just incorporate the likelihood into the weight.

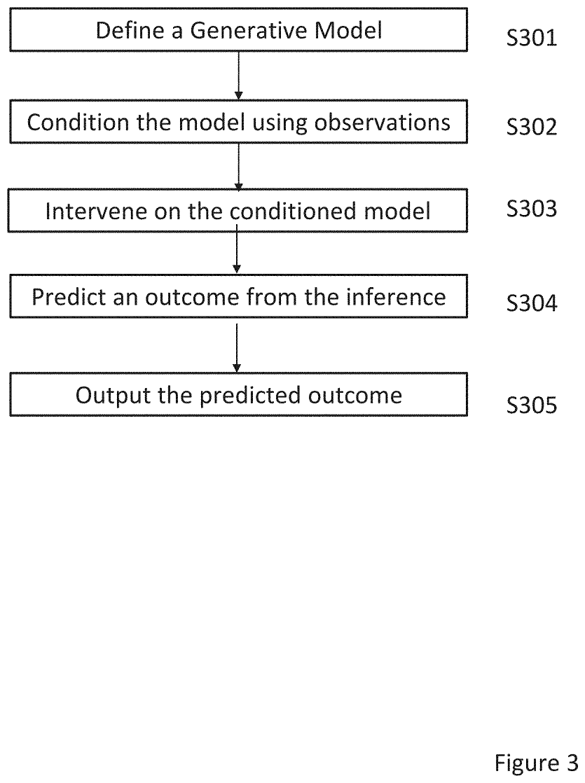

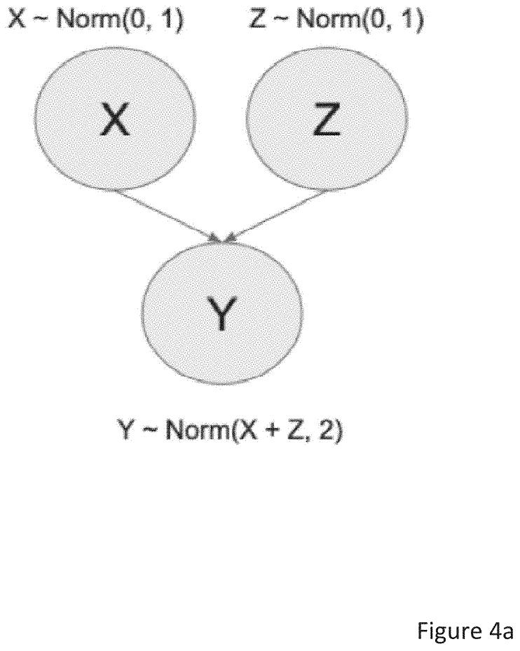

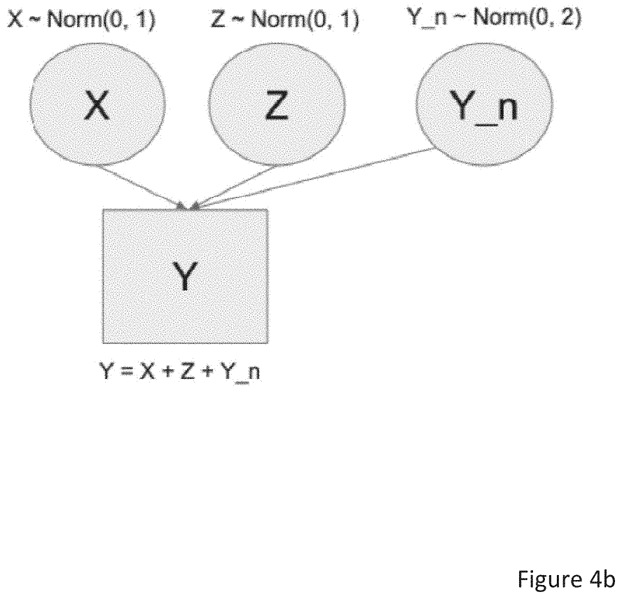

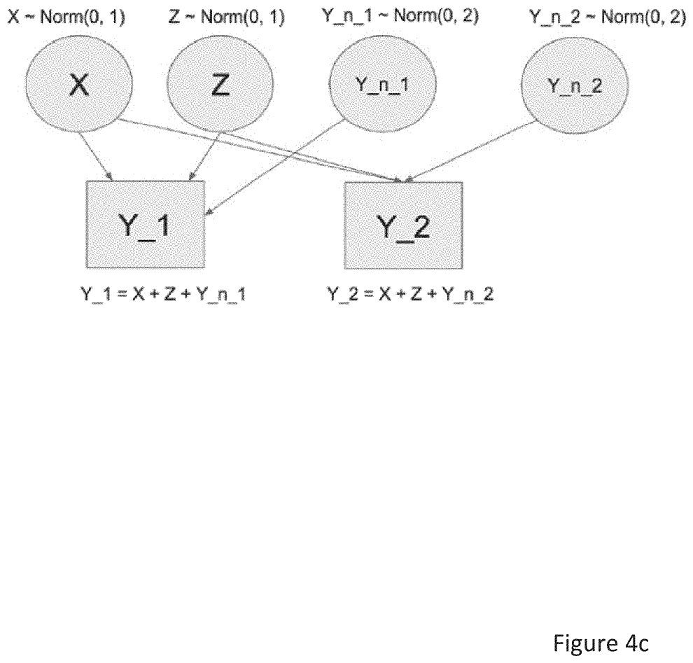

A counterfactual inference (the most complex version of a causal reasoning inference) method will be described with reference to FIG. 3 and with reference to the example model of FIGS. 4a, 4b and 4c.

In FIG. 3 in S301 the generative model is defined. The model may be a PGM or another probabilistic programming framework. In an embodiment, the generative model may be defined based on ERPs. The model may comprise both latent and observed variables. In another embodiment, instead of elementary random procedures, more complex random procedures can be used, e.g. exchangeable random procedures.

In defining the generative model, the prior and proposal distributions for any latent variables are defined. For observed variables, the conditional prior may be defined. The observed and latent variables may be defined using an elementary random procedure (ERP). There are many examples of ERPs known. For example, the Normal ERP is an ERP for the normal distribution. In another embodiment, other (not elementary; e.g. exchangeable) probabilistic procedures can be used.

In the example of FIG. 4a, the model is a simple continuous model comprising of one observed variable Y and two latent variables X and Z. The probabilistic program to define this model specifies the priors and proposals for the latent variables X and Z. The probabilistic program also specifies the conditional prior for Y. The probabilistic program defines these parameters using an ERP. In an example, the ERP may be for the Normal distribution. Below is example pseudo code used to define the model.

Although the model of FIG. 4a is a simple example, it will be appreciated that PGMs can be significantly complex. The abstraction of the model using ERPs assists in making the inference techniques more computationally efficient because the inference engine can perform optimisations on the structure of the network.

In S302, observations are added to the model. In the example of FIG. 4, there may be evidence that shows that Y=3, i.e. Y is observed to be 3. This observation may be added to the model.

In S303, the intervention is performed.

In S304, the prediction is performed. Importance sampling is used and thus, if the user selects an appropriate proposal, the samples are more relevant to the evidence, therefore, there is reduced variance in the output prediction (S305).

In S305, a predicted outcome may be output. For example, using the results of observational inference, the expected value of X if it is observed that Y=3, i.e. E_{Y=3}[X], may be calculated.

For computing counterfactual inference, the generative model must follow a structure similar to a structured causal model (SCM). Pearl defines a causal model as an ordered triple [symbol] U, V, E [symbol] , where U is a set of exogenous variables whose values are determined by factors outside the model; V is a set of endogenous variables whose values are determined by factors within the model; and E is a set of structural equations that express the value of each endogenous variable as a function of the values of the other variables in U and V.

The model of FIG. 4a is not a structured causal model. To make the model of FIG. 4a a structured causal model, the noise random variable Y_n needs to be separated from Y, as shown the model of FIG. 4b. Note that to make it a structural causal model by book, both the X and Z variables should be separated into exogenous and endogenous variables such that do operation can be performed on the endogenous variables. However, for this example, they are treated as one.

The probabilistic program to define this model specifies the priors and proposals for the latent variables X and Z. The probabilistic program also specifies the priors and proposals for the separated noise value Y_n. The probabilistic program defines these parameters using an ERP. In an example, the ERP may be for the Normal distribution.

In defining the noise variable Y_n, the observed value, e.g. Y=3, must be used as part of the proposal for the noise variable.

The probabilistic program to define this model also specifies the conditional prior for Y. However, as the noise variable Y_n is separated out from Y (to make this model a structured causal model), it needs to be ensured that the value of Y is proposed exactly to match the observed value of Y (if there is the observed value). Such proposal is necessary because it allows the avoidance of an intractable amount of rejections (or, in terms of the importance sampling, the zero weights) when a variable does not match its observed value.

To allow efficient inference, and to save computational and memory resource, a new class of ObserveableERPs (or ObserverableRPs that stand for Observable Random Procedures in general), e.g. ObservableNormalERP are introduced. Such ERP can be observed (i.e. have evidence provided), but it also explicitly creates a noise random variable inside the probabilistic programming execution trace as shown on FIG. 4b. When a probabilistic programming inference engine receives an observation, it forces (by proposing with probability 1.0) a value of the noise variable (e.g. Y_n on the Figure) to make the result variable (e.g. Y on the Figure) to match the value of the observation.

FIG. 4c shows a further representation where the noise is shown for two queries. It is important to note that if someone does not use an ObservableERP for the model in FIG. 4b but instead they try to run a counterfactual query on the model in FIG. 4a directly (without an explicit noise variable), then the inference actually will be performed according to the model in FIG. 4c, not according to the model in FIG. 4b as it would be desired.

The noise variable separation is done to ensure that in the abduction step of the counterfactual inference (i.e. in the step when the posterior distribution is calculated over all latents), the posterior is computed over the noise as well. To understand that better, it is important to understand that in counterfactual inference the minimum change to the world for calculating our “imaginary what-if query” is of interest, and therefore the noise should be from the posterior as well.

In order to perform counterfactual inference in probabilistic programming, current probabilistic programming frameworks require a user to represent and generate two models for performing counterfactual inference. For example, in Pyro, third-party probabilistic programming platform, a first model is defined. Next, the model is conditioned with the observations to create a second model. The noise variables are then retrieved from the second (conditioned) model. These steps combined define the new abducted generative model. Only following these steps can the do operation be performed. This additional step not only adds to the complexity of the method but also affects the level of abstraction required for defining the model. Moreover, in existing frameworks to do the final prediction step, as S304 as described in FIG. 3, it is generally required to re-sample from the intervened posterior distribution, which leads to biases and more approximation errors. All of the above means that more computational resource and memory should be used in existing prior-art probabilistic programming frameworks.

To simplify the procedure, the presented method provides a probabilistic programming framework that allows a user to separately specify a model, then at least one of observations and interventions, and then select which inference to perform (i.e. observational-posterior, or interventional, or counterfactual). It is important that those things are separate from one another for abstraction purposes, and in the embodiment the user does not have to use probabilistic programming framework methods themselves to perform a specific inference query (e.g. a counterfactual query).

In the presented method, the observations are also properly propagated. That is, a model can first be defined with a “regular” e.g. normal distribution which will properly separate its noise, and then do normal OBSERVE(VARIABLE, VALUE), and the embodiment will do everything for the user. That is, the user can define models as they generally define them in probabilistic programming languages and they don't need to implement any specific model adjustments for counterfactual inference. However, that it is not the case in the prior art.

The above method of probabilistic programming significantly simplifies the way modelling and inference is performed. Firstly, programming languages are used to define the model, and then, as a next independent step, evidence (observations) is provided. Then as another independent step, it can be decided what inference to use (observational, or interventional, or counterfactual) and to perform the inference.

Therefore, only one generative model needs to be defined, then observation, interventional or counterfactual inference can be performed on that model. There is no requirement to provide further modifications to the model, or create a new model, to perform, for example, counterfactual inference, as is the case with current methods.

Hence, the presented method is an efficient framework to do modelling and inference, including interventional and counterfactual inference.

More details of how the proposed method is used for interventional inference is now presented.

To perform interventional queries using importance sampling, a representation of the model with interventions is used (i.e. with ignored incoming edges to the intervened variables and such that those variables are intervened to specific values (i.e. just treated as fixed hyperparameters to their descendents) with values), and then the importance sampling algorithm is performed in that representation of the model.

In probabilistic programming, to generate N samples from an interventional query (W|do(D=d),E) over a probabilistic program that represents a model M, the following steps are performed:

1. Calculate the program first time to record what variables are intervened and what variables are observed, and with what values in both cases.

2. To execute that program N more times to generate samples {sᵢ}.

3. During each execution, you need to make sure that for variables in D, you don't compute their values from the model but instead you force that value to the value from d.

4. As usual with importance sampling in probabilistic programming, you need to sample X\D ∪ E from a proposal, incorporate the prior and proposal likelihoods of X\D ∪ E into the weights, as well as the likelihoods of observed values E.

5. For each sample sᵢ, we return with W ⊆ X sample weights. These are the predictions.

The memory and computational complexity of importance sampling for interventional queries is the same as the complexity of importance sampling for “observational” queries as each program needs to be evaluated once. Note that FIG. 3 represents counterfactual queries. For interventional queries, in FIG. 3 step S303 is performed before step S302; for pure observational queries, step S303 is skipped by the nature of that inference query.

More details of how the proposed method is used for counterfactual inference is now presented.

To recap, for the counterfactual query P(W′|E,do(D=d)), firstly the posterior distribution (abduction step) is obtained, and then do operations are performed (i.e. the intervention step) on it, so that predictions can be generated from there (i.e. the prediction step). These steps can be separated into two main parts:
1. Using importance sampling to obtain a representation of the posterior distribution P(W|E). The key idea is that the posterior distribution can be represented approximately in the form of samples s₁ . . . sₙ and their weights s₁ . . . sₙ. That is, the set of tuples {sᵢ, wᵢ} is a valid approximate representation of a posterior distribution P(W|E).
2. Do do operations on that representation. In other words, do( . . . ) can be performed on each sample sᵢ such that i the effect of doing do on every variable can be propagated to every other variable that depends on it recursively. By doing so, a new set of tuples {s′ᵢ, wᵢ} is obtained. That set exactly represents the intervened samples from a posterior distribution (under the approximations of the importance sampling). That way, the following can be computed:

$${E}_{{W}^{′} \vert E , d α ( D ∞ d )} = \frac{Σ f ( {s}_{i}^{′} ) \times {w}_{i}}{Σ {w}_{i}} .$$

To compute samples from counterfactual query in probabilistic programming settings involves the following:
1. Calculate the program first time to record what variables are observed and what variables are intervened, and with what values in both cases.
2. To execute that program N more times without any intervention,
3. As usual with importance sampling in probabilistic programming, you need to sample X\E from a proposal, incorporate the prior and proposal likelihoods of X\E into the weights, as well as the likelihoods of observed values E.
4. After that, N new samples based on samples {sᵢ} need to be generated. For each sample sᵢ, the program needs to be re-evaluated but instead of sampling random variables, for each random variable a value that was sampled for that random variable is reused, unless that variable is in D or it is any descendent variable of any variable in D. If the variable is in D, the variable is forced to be the value of d. If the variable is a descendent (direct or indirect) of any variable in D, then it needs to be re-evaluated.

Note a nuance about re-evaluating the variables that are descendents of any variables in set D. Following the convention suggested by Pearl et al. (e.g. see Causality), do operation can happen only on endogenous variables. Also, following a similar principle, any variable that is a descendent (direct or indirect) of an intervened variable should also be an endogenous variable. That is one of the requirements of working with structural causal models. Note that in general in probabilistic programming a variable that is a descendant of any variable in D can be a random variable with some hyperparameters, in other words such variable is both an exogenous variable (defined by its own randomness (e.g. noise) that is not expressed as a separate part of the model and hence breaks the assumptions of structural causal models) and an endogenous variable (by the virtue of having hyperparameters that depend on other variables in the model).

There might be at least three options to cope with this situation:

1. Formulate queries by ensuring the model is a strict structural causal models and all the queries are appropriate.

2. Introduce checks and restrictions in the probabilistic programming platform to make sure that only endogenous can be observed or intervened, and that any descendents of endogenous variables are also endogenous variables.

3. A “heretical” approach is to use this hidden randomness for the sake of using the prior conditional distributions for such variables. E.g., it can be useful if we assume that in the counterfactual query the noise is defined by it prior distribution even at the counterfactual's intervention step. However, it is only a hack due to the implementation, and if somebody would like to model something like that, it might be the best to introduce proper constructions for that (e.g. by specifying what variables should be resampled from their prior in the interventional part of a counterfactual query; or by adjusting the model and doing partial counterfactual queries as shown in one of the examples below).

Based on the above steps, it is noted that 2N+1 evaluations of the program are needed. s′ᵢ can also be calculated immediately after sᵢ is calculated rather than firstly calculating all {sᵢ} and only then calculating {sᵣᵢ}. Hence, the memory and computational complexity of importance sampling for counterfactual queries is the same in terms of O-notation as the complexity of importance sampling for “observational” queries as we need to evaluate each program twice. However, it takes 2 times more in terms of constant factor. Further optimisations can be made to track the dependency of a complex model graph to make sure that for computing only the necessary sub-parts of a program/model are re-evaluated. As with any importance sampling methods in general, counterfactual queries can be done in parallel. As for the memory optimisations, instead of keeping full samples in the memory, all but predicted values for W′ can be discarded (or, even further, only statistics of interest need be accumulated, e.g. a mean or variance).

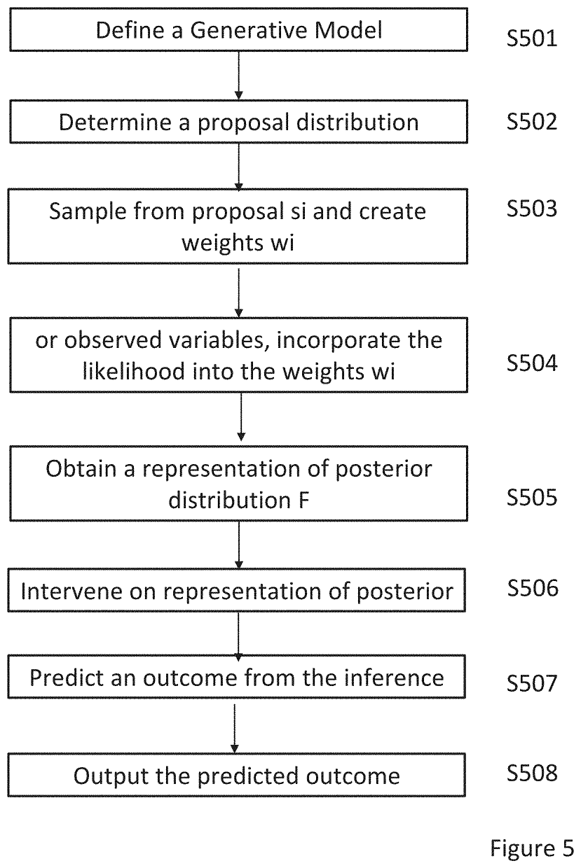

FIG. 5 details an embodiment of the method of FIG. 3 when counterfactual inference is selected. In S501, the generative model is defined as a model comprising observed and latent variables. This involves separating out the noise term from any observed variables, such as separating Y_n from Y in FIG. 4b. In one embodiment, the latent variables should be separated into exogenous and endogenous variables. In other embodiment, the separation is not necessary because it operates on Bayesian networks or generative models, in general.

In S504, observations are taken into the account, for example Y=3.

In S506, interventions will be used.

In S502-S505, the abduction step of the counterfactual inference is performed, if chosen, using importance sampling. In this step a representation of the posterior distribution is obtained {si, wi} for all latent variables. The representation of the posterior distribution may include the following steps:
a) For latent variables (exogenous),
   a. determine a proposal distribution
   b. sample from proposal, si
   c. create weights wi
b) For observed variables, incorporate the likelihood into the weight
c) Obtain a representation of the posterior distribution {si, wi}

Then, in S506 do operation is performed on the samples (which represent the posterior) and therefore receive intervened samples {si′} based on {si}. Then the quantities of interest of the counterfactual inference (e.g. expected values) are predicted in S507 by using {si′} and weights {wi}. In S508, the output is produced.

In another embodiment, a combination of categorical and continuous variables can be used with the same algorithm.

Furthermore, because of the separation and abstraction of the steps, in some embodiments improvements on the inference (i.e. speed, optimisations, etc.) can be implemented. In other words, optimisations can be performed based on the fact that it is known what inference is needed to run given a model.

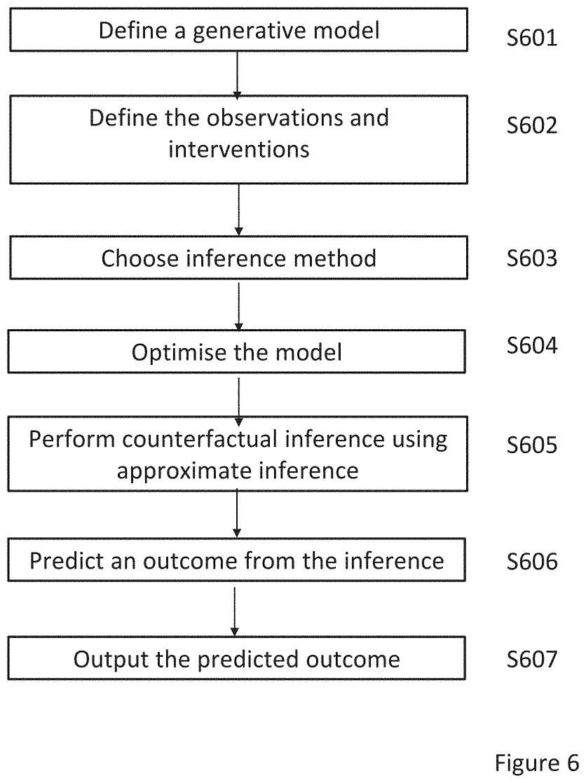

With reference to FIG. 6, following the definition of the model in S601, and the adding of observations and interventions in S602, and selecting an inference method in S603, static analysis is performed on the model in S604. For example, by considering the specifics of the query (observational, interventional or counterfactual) and inference scheme, one can exploit several optimizations. Probabilistic programming engines do this natively by defining these optimizations as part of their inference procedure. First, a counterfactual importance sampling procedure, given a query, can automatically reduce the size of the proposal distribution by placing a proposal only over variables that are not independent of the variable(s) of interest, given the evidence. For example, in graphical terms, the evidence may form a blanket that d-separates the node of interest from much of the graph. Second, in our system there is no double-sampling; we sample once from the proposal distribution and estimate the value of interest online. This also saves an additional approximation error, as in other implementations one would sample from the proposal and then also sample from the posterior. Also, we also save on memory (and possibly time) as we need not define any new model structures beyond the original and as we don't need to pass any models between stages of the inference. In addition to that, in another embodiment, a static analysis can be done to identify the part of the program execution trace that can be ignored and does not need to be re-evaluated as everything else below it has been intervened and thus there is no further dependence on ancestor values. In addition to static analysis, in another embodiment a more sophisticated dynamic analysis can be performed during the program execution with complex, probabilistic program control flows. Finally, for the intervention steps, in another embodiment technology called “Copy-on-write” can be used to avoid duplication of data structures for the intervened part of the model such that only the parts of the model that have been changed are copied; this improves the memory efficiency as well.

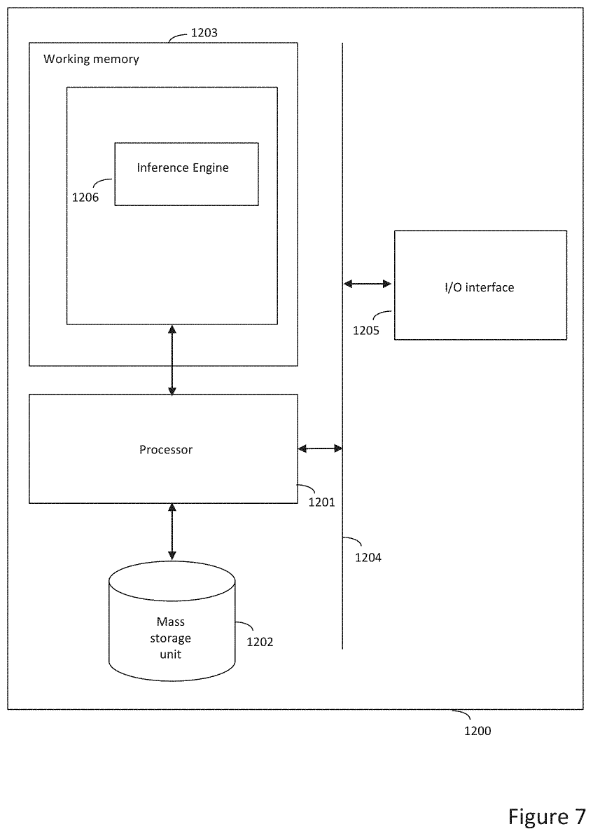

While it will be appreciated that the above embodiments are applicable to any computing system, an example computing system is illustrated in FIG. 7, which provides means capable of putting an embodiment, as described herein, into effect. As illustrated, the computing system 1200 comprises a processor 1201 coupled to a mass storage unit 1202 and accessing a working memory 1203. As illustrated, an inference engine 1206 obtained by the described method is represented as software products stored in working memory 1203. However, it will be appreciated that elements of the inference engine 1206 described previously, may, for convenience, be stored in the mass storage unit 1202.

Depending on the use, the inference engine 1206 may be used with a chatbot, to provide a response to a user question.

Usual procedures for the loading of software into memory and the storage of data in the mass storage unit 1202 apply. The processor 1201 also accesses, via bus 1204, an input/output interface 1205 that is configured to receive data from and output data to an external system (e.g. an external network or a user input or output device). The input/output interface 1205 may be a single component or may be divided into a separate input interface and a separate output interface. Thus, execution of the inference engine 1206 by the processor 1201 will cause embodiments as described herein to be implemented.

The inference engine 1206 can be embedded in original equipment, or can be provided, as a whole or in part, after manufacture. For instance, inference engine 1206 can be introduced, as a whole, as a computer program product, which may be in the form of a download, or to be introduced via a computer program storage medium, such as an optical disk. Alternatively, modifications to existing causal discovery model software can be made by an update, or plug-in, to provide features of the above described embodiment.

The computing system 1200 may be an end-user system that receives inputs from a user (e.g. via a keyboard) and retrieves a response to a query using the inference engine 1206 adapted to produce the user query in a suitable form. Alternatively, the system may be a server that receives input over a network and determines a response. Either way, the use of the inference engine 1206 may be used to determine appropriate responses to user queries, as discussed with regard to FIG. 1.

Implementations of the subject matter and the operations described in this specification can be realized in digital electronic circuitry, or in computer software, firmware, or hardware, including the structures disclosed in this specification and their structural equivalents, or in combinations of one or more of them. Implementations of the subject matter described in this specification can be realized using one or more computer programs, one or more modules of computer program instructions, encoded on computer storage medium for execution by, or to control the operation of, data processing apparatus. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., multiple CDs, disks, or other storage devices).

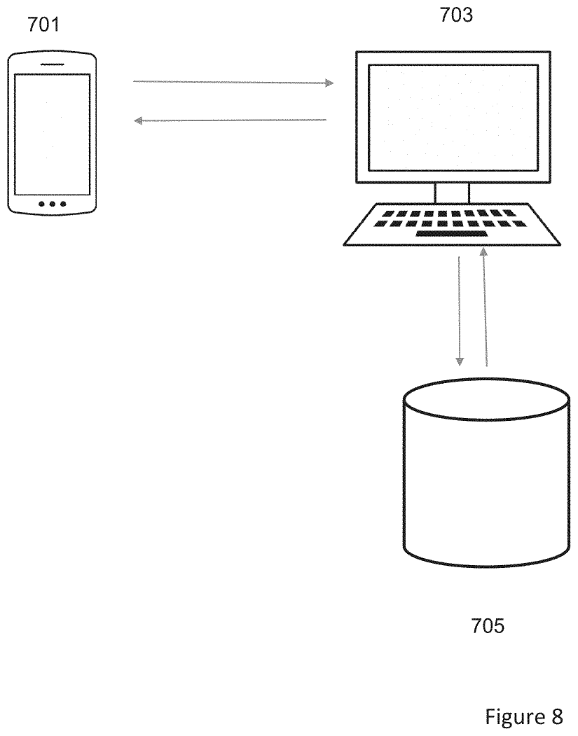

FIG. 8 shows a further example of the apparatus. The inference engine may be provided on a computer 703. The computer 703 is linked to memory 705. The memory stores the PGM. As noted above, only one PGM needs to be stored for the three types of inference.

In this embodiment, the inference query is input via a mobile device 701. The inference query is processed by the computer without any further information required from the mobile 701.

While certain embodiments have been described, these embodiments have been presented by way of example only, and are not intended to limit the scope of the inventions. Indeed the novel methods and systems described herein may be embodied in a variety of other forms; furthermore, various omissions, substitutions and changes in the form of methods and systems described herein may be made without departing from the spirit of the inventions. The accompanying claims and their equivalents are intended to cover such forms of modifications as would fall within the scope and spirit of the inventions.

1. A computer implemented method of performing inference on a generative model,
wherein the generative model in a probabilistic program form, said probabilistic program form defining variables and probabilistic relationships between variables, the method comprising:
providing at least one of observations or interventions to the generative model;
selecting an inference method, wherein the inference method is selected from one of: observational inference, interventional inference or counterfactual inference;
performing the selected inference method using an approximate inference method on the generative model; and
outputting a predicted outcome from the results of the inference,
wherein approximate inference is performed by inputting an inference query and the model, observations, interventions and inference query are provided as independent parameters such that they can be iterated over and varied independently of each other.

2. A method according to claim 1, wherein approximate inference is performed in an inference engine and each inference query is fully performed as just one single request to the inference engine.

3. A method according to claim 1, wherein the approximate inference method is importance sampling.

4. A method according to claim 1, wherein the generative model expresses noise as explicit random variables.

5. A method according to claim 1, wherein performing observational inference comprises:
retrieving said generative model; and
weighting the prior space of the generative model by the likelihood that is calculated on the observed probabilistic procedures.

6. A method according to claim 1, wherein performing interventional inference comprises:
retrieving said generative model;
representing said generative model with intervened variable; and
weighting the prior space of the generative model representation by the likelihood that is calculated on the observed probabilistic procedures.

7. A method according to claim 1, wherein performing counterfactual inference comprises:
retrieving said generative model;
weighting the prior space of the generative model by the likelihood that is calculated on the observed probabilistic procedures; and
performing intervention on a representation of that model and predicting the variables of interest.

8. A method according to claim 7, wherein during the weighting the prior space of the generative model by the likelihood that is calculated on the observed probabilistic procedures, the noise distributions of observed probabilistic procedures are represented.

9. A method according to claim 2, wherein the generative model is provided on a first server and the inference query is input at a location separate from the first server, wherein the inference query is sent as a single request from the said location to the first server and the server returns the result of the inference as a single message to the said location.

10. A method according to claim 8, wherein the result of the inference is the calculated inference query result.

11. A method according to claim 9, wherein the inference query is input via a mobile device.

12. A method according to claim 1, further comprising:
performing static analysis on the generative model, observations and interventions, given inference query types, to optimise the inference method.

13. A method according to claim 1, further comprising:
performing a dynamic analysis on the generative model, observations and interventions, given inference query types, to optimise the inference method.

14. A system adapted to perform inference on a generative model,
the system comprising a processor and a memory, the generative model being stored in a probabilistic program form in said memory, said probabilistic program form defining variables and probabilistic relationships between variables, the processor being configured to:
provide at least one of observations or interventions to the generative model;
allow selection of an inference method, wherein the inference method is selected from one of: observational inference, interventional inference or counterfactual inference;
perform the selected inference method using an approximate inference method on the generative model; and
output a predicted outcome from the results of the inference;
wherein approximate inference is performed by inputting an inference query and the model, observations, interventions and inference query are provided as independent parameters such that they can be iterated over and varied independently of each other.

15. A non-transitory computer medium carrying computer readable instructions that when run on a computer will cause the computer to perform the method of claim 1.
